(19) United States
(12) Patent Application Publication
(10) Pub. No.: US 2026/0158228 A1
(43) Pub. Date: Jun. 11, 2026

(54) DRY-POWDER INHALER

(71) Applicant: APTAR FRANCE SAS, LE NEUBOURG (FR)

(72) Inventors: Youcef BELKHEROUF, EVREUX (FR); Sébastien MICHAUX, LE PETIT QUEVILLY (FR); Jonathan TOURNAIRE, GRAND COURONNE (FR); Franck POULLAIN, LA HAYE MALHERBE (FR); Ludovic PETIT, VITOT (FR)

(73) Assignee: APTAR FRANCE SAS, LE NEUBOURG (FR)

(21) Appl. No.: 19/103,898

(22) PCT Filed: Aug. 16, 2023
(86) PCT No.: PCT/FR2023/051269

§ 371 Date: Feb. 14, 2025

(30) Foreign Application Priority Data
Aug. 18, 2022 (FR) FR2208363

(51) Int. Cl. A61M 15/00

(52) U.S. Cl. CPC A61M 15/0028; A61M 2202/064

(57) ABSTRACT

A dry-powder inhaler having a body containing a chamber and a mouthpiece with a dispensing orifice, a reservoir containing a dose of dry powder and having a lateral outlet opening, the reservoir movable between an non-loaded position and a loaded position, the diameter of the reservoir being less than the diameter of the chamber, so that, in the loaded position, the reservoir can move in the chamber. The body has tangential channels bringing a flow of air tangentially into the chamber; and an actuating member for moving the reservoir from its non-loaded position towards its loaded position. The reservoir has a closure element displaceable and/or deformable relative to the reservoir between closed and open positions of the lateral outlet opening, the closure element being in the closed position when the reservoir is in the non-loaded position, and in the open position when the reservoir is in the loaded position.

The present invention relates to a dry-powder inhaler.

Dry-powder inhalers are well known in the prior art. Various types exist.

A first type of multidose inhaler contains a reservoir receiving multiple doses of powder, the inhaler being provided with dosage means, making it possible, on each actuation, to separate out one dose of this powder from the reservoir in order to bring it into an expulsion duct so that it can be dispensed to the user. Such devices require complicated and therefore expensive dosage means, and dosage accuracy and reproducibility are not guaranteed. In addition, there are risks of contamination of the powder disposed in the reservoir as the inhaler is being used.

Another type of multidose inhaler consists of providing several individual reservoirs each containing one dose of powder, then opening one of those reservoirs each time the inhaler is actuated. That implementation ensures that the powder is better sealed, since each dose of the powder is only exposed to the atmosphere at the moment when it is expelled. In order to produce such sets of individual reservoirs, different variations have already been proposed, such as blister strips or disks. With regard to opening the individual reservoirs, peeling off or unsticking the closure layer of the blisters has been proposed. That suffers from the disadvantage of difficulty in controlling the forces to be applied in order to guarantee full opening, without running the risk of opening the next reservoir, particularly if the opening means need to be actuated by inhalation. Another solution is to pierce the closure membrane of a blister on each actuation, which requires complicated and therefore expensive piercing means.

In addition, the multidose inhalers described above suffer from the problem of not guaranteeing optimal dispensing effectiveness, with a significant portion of the dose effectively penetrating into the user's lungs in order to have a beneficial therapeutic effect.

In order to make devices less complicated and thus less expensive, single-dose inhalers have been proposed which comprise a single individual reservoir, such as a blister or a capsule, which has to be loaded into the inhaler just before it is used. The advantage of such single-dose devices is that it is not necessary to store all of the doses inside the device, so that the dimensions thereof can be reduced. In contrast, it is more complicated for the user to use, since they are obliged to load a blister or a capsule into the inhaler before each use. Furthermore, other disadvantages specific to these single-dose inhalers exist. Thus, a blister or capsule does not allow large doses, typically greater than 100 mg, to be dispensed. In addition, in particular as regard capsules or blisters to be pierced, opening requires the use of complicated piercing means, and also risks getting residues of pierced membrane in the powder which is dispensed. Furthermore, the emptying performance of the reservoir is not always optimal, since a portion of the powder could remain inside the reservoir during inhalation.

In an attempt to overcome these disadvantages, document WO 98/26828 proposes a single-dose inhaler comprising a cylindrical reservoir provided with a lateral opening, disposed in a cylindrical chamber with a larger diameter, in a manner such that during inhalation, the reservoir is displaced in the chamber in accordance with an orbital movement, which causes the powder to be expelled. This implementation effectively makes it possible to improve the efficiency of dispensing, with substantially complete emptying of the reservoir and a larger portion of the dose which penetrates effectively into the lungs. Nevertheless, a disadvantage with this device is that the lateral opening of the reservoir must be opened before the reservoir is placed in the device, which presents a risk of losing powder during this placing, or contamination of the latter, in particular if the device is not used quickly after placing the reservoir.

Documents WO2008001132A1, WO03075988A1, US2014182587A1 and WO2013008037A1 describe other devices of the state of the art.

An object of the present invention is to provide a powder inhaler that does not have the above-mentioned drawbacks.

In particular, an object of the present invention is to provide an inhaler of this type that improves dispensing efficiency, by allowing almost all of the powder contained in the reservoir to be dispensed and by increasing the portion of the dose that will reach the user's lungs.

Another objective of the present invention is to provide an inhaler of this type which limits or even avoids the risks of contamination and/or pollution of the powder. Another objective of the present invention is to provide an inhaler of this type which reduces the complexity of use for the user and which guarantees better safety in use.

Another objective of the present invention is to provide an inhaler of this type which is simple and inexpensive to manufacture and to assemble.

The present invention thus provides a dry-powder inhaler comprising:
a body containing a chamber and a mouthpiece provided with a dispensing orifice through which the user inhales,
a reservoir containing a dose of dry powder to be inhaled and comprising a lateral outlet opening, said reservoir being movable between an non-loaded position, in which it is at least partially disposed outside said chamber, and a loaded position, in which it is entirely disposed in said chamber, the diameter of said reservoir being less than the diameter of said chamber, so that, in the loaded position, said reservoir can move in said chamber, said body comprising tangential channels adapted to bring a flow of inhalation air tangentially into said chamber, which, during inhalation, generates an orbital movement of said reservoir in said chamber, in the loaded position,
an actuating member for moving said reservoir from its non-loaded position towards its loaded position,
said reservoir comprising a closure element which is displaceable and/or deformable with respect to said reservoir between a closed position of said lateral outlet opening and an open position of said lateral outlet opening, said closure element being in the closed position when said reservoir is in the non-loaded position, and being in the open position when said reservoir is in the loaded position.

Advantageously, said closure element comprises an asymmetrical radial projection.

Advantageously, said closure element comprises at least one second radial projection which extends axially over part of the axial height of said closure element.

Advantageously, said reservoir is formed by an upper reservoir portion and a lower reservoir portion, which are assembled after filling with the dose of powder.

Advantageously, said lower reservoir portion comprises said lateral outlet opening.

The present invention also provides a dry-powder inhaler comprising:
a body containing a chamber and a mouthpiece provided with a dispensing orifice through which the user inhales,
a reservoir containing a dose of dry powder to be inhaled and comprising a lateral outlet opening, said reservoir being movable between a non-loaded position, in which it is at least partially disposed outside said chamber, and a loaded position, in which it is entirely disposed in said chamber, the diameter of said reservoir being less than the diameter of said chamber,
an actuating member for moving said reservoir from its non-loaded position towards its loaded position,
said reservoir comprising an upper reservoir portion provided with an upper lateral opening and a lower reservoir portion provided with a lower lateral opening, said upper reservoir portion being axially movable relative to said lower reservoir portion, said upper and lower lateral openings being axially offset when said reservoir is in the non-loaded position, and being aligned to form an open lateral opening when said reservoir is in the loaded position.

Advantageously, said body comprises two body portions, an upper body portion comprising the mouthpiece and a lower body portion comprising a cylindrical sleeve, said chamber being defined by these two body portions fixed to one another.

Advantageously, said mouthpiece comprises a grid upstream from said dispensing orifice.

Advantageously, said chamber is disposed between an inhalation air inlet and said mouthpiece.

Advantageously, said reservoir contains a dose of powder greater than 50 mg, in particular greater than 100 mg.

Advantageously, the volume occupied by said reservoir in said chamber in the loaded position is greater than 50% of the volume of said chamber.

Advantageously, the radial diameter of said reservoir is greater than its axial height.

Advantageously, said actuating member is axially displaceable with respect to said body in order to displace said reservoir from its non-loaded position towards its loaded position.

Advantageously, said actuating member comprises at least one oblique groove receiving at least one respective lug formed on said body or on an element which is secured to said body, in a manner such that during its axial displacement, said actuating member implements a rotation in said body.

These features and advantages and others of the present invention will appear more clearly during the following detailed description, made in reference to the accompanying drawings, given as non-limiting examples, and in which

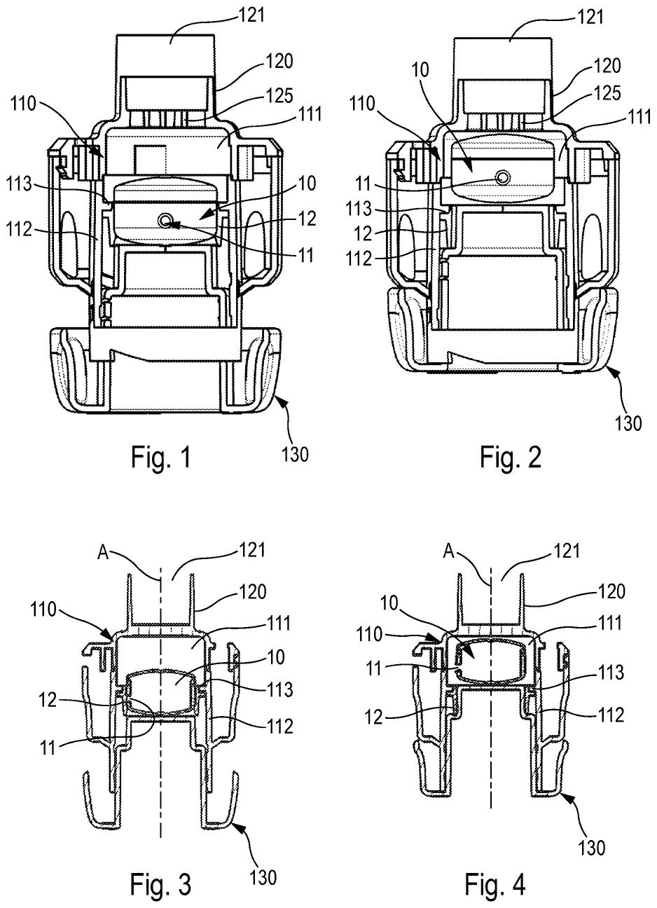

FIG. 1 is a diagrammatic cross-sectional view of an inhaler in accordance with a first advantageous embodiment, in the non-loaded position,

FIG. 2 is a view similar to that of FIG. 1, in the loaded position,

FIG. 3 is a diagrammatic partial in cross-sectional view according to another section plane of dispensing device of the FIG. 1, in the non-loaded position,

FIG. 4 is a view similar to that of FIG. 3, in the loaded position,

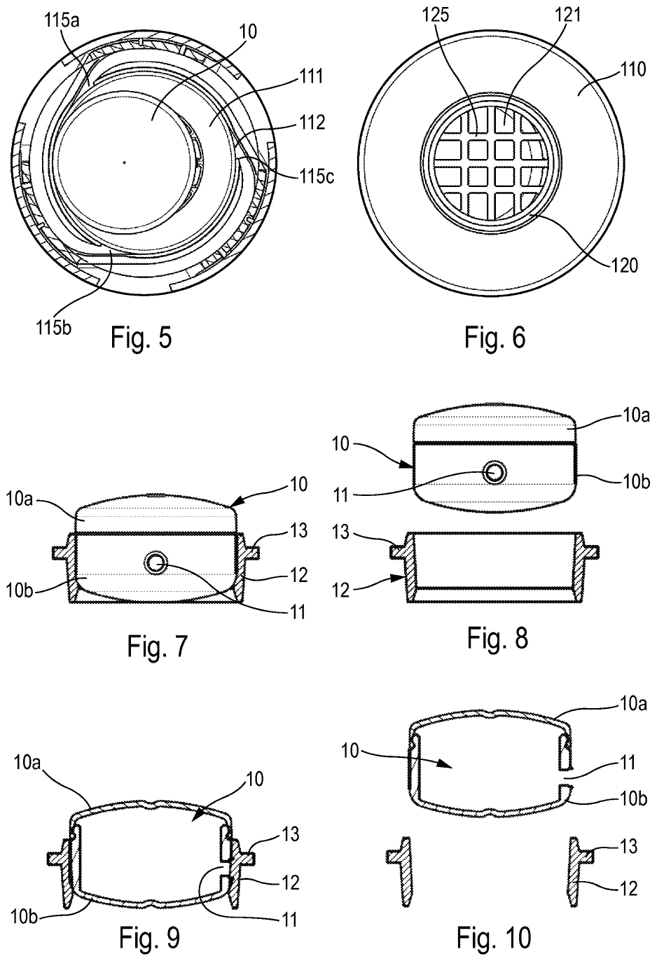

FIG. 5 is a diagrammatic view from below of the reservoir in the chamber, in the loaded position,

FIG. 6 is a diagrammatic view from above of the mouthpiece.

FIG. 7 is a schematic, vertical cross-sectional view of the reservoir, in the non-loaded position,

FIG. 8 is a view similar to that of FIG. 7, in the loaded position,

FIG. 9 is a diagrammatic view similar to that of FIG. 7, from another viewing angle, in the non-loaded position,

FIG. 10 is a view similar to that of FIG. 9, in the loaded position,

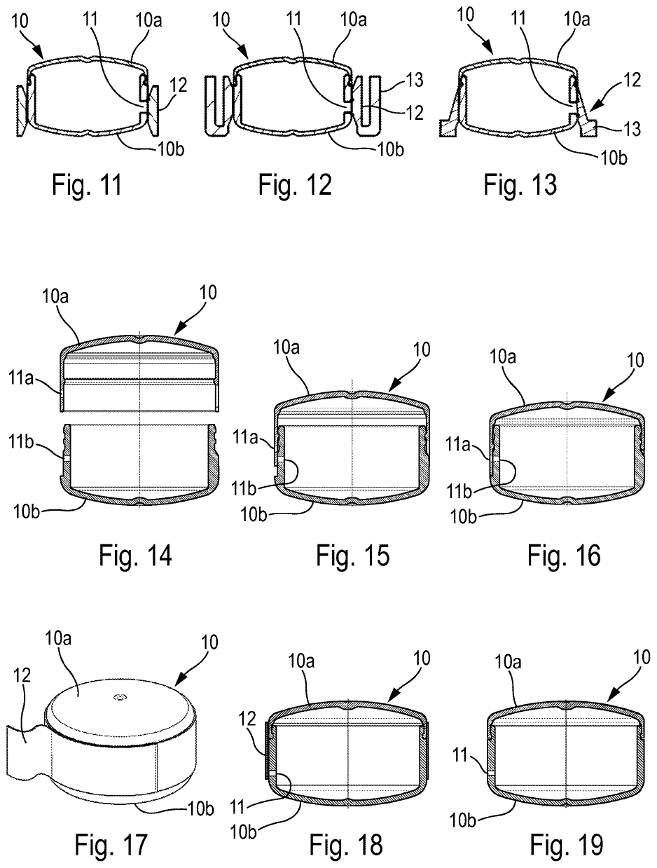

FIGS. 11 to 13 are diagrammatic, vertical section views of three other advantageous variant embodiments of the reservoir, in the non-loaded position,

FIGS. 14 to 16 are diagrammatic, vertical cross-sectional views of a reservoir in accordance with another embodiment, respectively before assembling, in the non-loaded position and in the loaded position,

FIG. 17 is a diagrammatic, perspective view of the reservoir showing the reservoir in accordance with still another embodiment, in the loaded position,

FIGS. 18 and 19 are diagrammatic, vertical cross-sectional views of the reservoir of FIG. 17, respectively in the non-loaded position and in the loaded position,

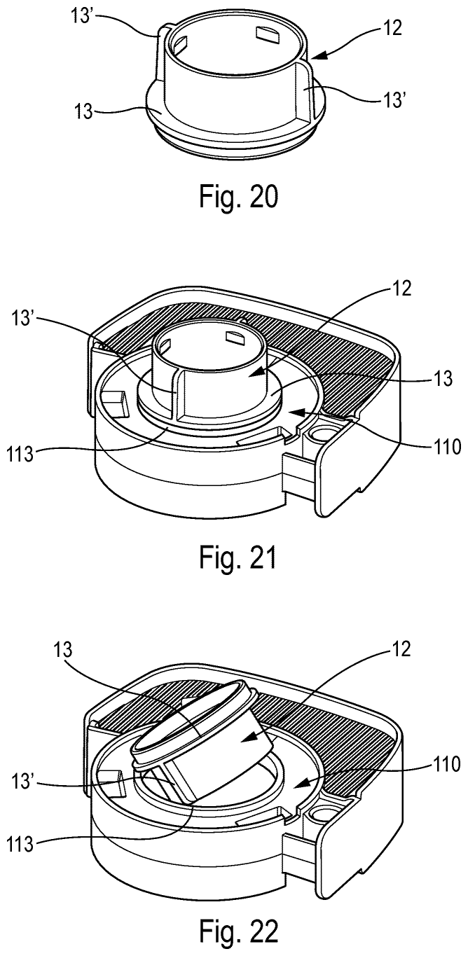

FIG. 20 is a diagrammatic perspective view of a closure element according to another advantageous variant,

FIG. 21 illustrates the possible assembly in the correct orientation of the closure element of FIG. 21, and

FIG. 22 illustrates the impossible assembly in the wrong orientation of the closure element of FIG. 21.

In the description below, the terms “upper”, “lower” and “lateral” are with respect to the upright position of the device shown in FIGS. 1 to 4. The terms “axial” and “radial” are relative to the longitudinal central axis A of the device shown in FIGS. 3 and 4.

FIGS. 1 to 10 show a first embodiment of the invention.

In this first embodiment, the inhaler comprises a body 110 containing a chamber 111 and a mouthpiece 120 provided with a dispensing orifice 121 through which the user inhales.

Advantageously, the body 110 is formed by two body portions, an upper body portion comprising the mouthpiece 120 and a lower body portion comprising a cylindrical sleeve 112, the chamber 111 being defined by these two body portions fixed to one another.

Advantageously, as can be seen in FIG. 6, the mouthpiece 120 includes a grid 125 upstream from the dispensing orifice 121.

Preferably, the chamber 111 is disposed between an inhalation air inlet and said mouthpiece 120, in a manner such that when the user inhales through the mouthpiece 120, the air flow will pass through said chamber 111.

The inhaler comprises a reservoir 10 containing a dose of dry powder to be inhaled and comprising a lateral outlet opening 11. This reservoir 10 is displaceable between an non-loaded position, in which it is at least partially disposed outside said chamber 111, and a loaded position, in which it is entirely disposed in said chamber 111.

The diameter of the reservoir 10 is less than the diameter of said chamber 111, in a manner such that in the loaded position, the reservoir 10 can be displaced in the chamber 111. Tangential channels 115a, 115b, 115c bring said flow of inhalation air tangentially into the chamber 111, thereby causing the reservoir 10 to move in the chamber 111 in an orbital manner, the reservoir 10 rotating on itself while rotating about the periphery of the chamber 111 along the side wall thereof. This movement will allow the powder contained in the reservoir 10 to be expelled, which is entrained by the flow of inhalation air towards the mouthpiece and the dispensing orifice.

Advantageously, the reservoir 10 may contain a dose of powder greater than 50 mg, in particular greater than 100 mg. Optionally, very large doses may be envisaged, in particular greater than 500 mg.

Advantageously, the volume occupied by the reservoir 10 in the chamber 111 in the loaded position is greater than 50% of the volume of the chamber 111.

Advantageously, the radial diameter of the reservoir 10 is greater than its axial height.

Advantageously, the reservoir 10 is formed by an upper reservoir portion 10a and a lower reservoir portion 10b, which are assembled after filling with the dose of powder.

The inhaler also comprises an actuating member 130 for displacing said reservoir 10 from its non-loaded position towards its loaded position. This actuating member 130 is axially displaceable with respect to the body 110 in order to push the reservoir 10 from its non-loaded position towards its loaded position.

Advantageously, the axial displacement of the actuating member 130 is produced by a rotation of the actuating member 130 in the body 110, for example by means of one or more lugs of the body 110 disposed in one or more oblique grooves of the actuating member 130.

According to the first embodiment of the invention represented on FIGS. 1 to 10, the reservoir 10 comprises a lateral outlet opening 11 closed by a closure element 12 when the reservoir 10 is not in its loaded position.

Advantageously, it is the lower reservoir portion 10b which comprises the lateral outlet opening 11.

This closure element 12 protects the powder contained in the reservoir until it is dispensed by actuating the inhaler.

Thus, according to a first aspect of the invention, the closure element 12 is displaceable and/or deformable with respect to the reservoir 10 between closed and open positions of the lateral outlet opening 11, said closure element 12 being in the closed position when said reservoir 10 is in the non-loaded position, and being in the open position when said reservoir 10 is in the loaded position.

Advantageously, in the non-loaded position, the closure element 12 abuts against a portion of the body 110 or is secured thereto, so that when the reservoir 10 is displaced towards its loaded position, the closure element 12 remains locked in the body, thereby opening the lateral outlet opening 11.

In the example of FIGS. 1 to 10, the closure element 12 comprises a radial projection 13 which cooperates with a shoulder 113 of the cylindrical sleeve 112.

Advantageously, the radial projection 13 is disposed to one side of the closure element 12, so that the closure element is not symmetrical. Thus, when the closure element 12 is in the closed position on the reservoir 10, the latter is not symmetrical either; this forms a foolproof key for the insertion of the reservoir 10 into the body 110 of the inhaler, this insertion only being possible in a single orientation of the reservoir 10. This is particularly advantageous when the external shape of the reservoir 10 is substantially symmetrical, but the lateral outlet opening 11 is not disposed at the axial centre of the reservoir 10, as in the examples in the figures. In fact, in this case, it is desirable to correctly orientate the reservoir 10 in the inhaler for optimal operation, and the asymmetrical shape of the closure element makes it possible to fulfil this function.

FIGS. 20 to 22 show another variant in which the closure element 12 comprises at least one second radial projection 13′ that extends axially over a portion of the height of the closure element 12, advantageously between an axial edge and said radial projection 13. This makes it possible to avoid any risk of assembling the reservoir 10 upside down, as shown in FIGS. 21 and 22. In FIG. 21, the orientation is correct, and the reservoir 10+ closure element 12 assembly can be assembled in the body 110, whereas in the orientation of FIG. 22, this assembly is made impossible by the presence of the second radial projection 13′. In the example shown, there are two second radial projections 13′ that are diametrically opposed, but any number is possible.

Clearly, if the reservoir 10 is symmetrical, with the lateral outlet opening 11 centred axially, the closure element 12 does not need to be asymmetrical. In contrast, a closure element 12 forming a symmetrical unit with the reservoir 10 would allow the user to insert this unit into the inhaler in both orientations, without any risk of malfunction or less effective powder distribution.

Variant embodiments of the closure element 12 are represented in FIGS. 11 to 13.

FIGS. 14 to 16 illustrate a second embodiment. Here, the closure element is formed directly by the upper reservoir portion 10a, and it is not necessary to provide a separate closure element.

In this second embodiment, the upper reservoir portion 10a is provided with an upper lateral opening 11a and the lower reservoir portion 10b is provided with a lower lateral opening 11b. The upper reservoir portion 10a is axially movable relative to said lower reservoir portion 10b, with said upper and lower lateral openings 11a, 11b being axially offset when said reservoir 10 is in the non-loaded position, and being aligned to form an open lateral opening when said reservoir 10 is in the loaded position.

FIGS. 17 to 19 illustrate a third embodiment. Here, the closure element 12 is formed by a flexible strip fixed or glued around the reservoir 10 to close the lateral outlet opening 11. In this case, the inhaler comprises means for fixing the free end of said flexible strip, so that when the reservoir 10 is moved from its non-loaded position towards its loaded position, the closure element 12 is removed from the reservoir 10. For example, it is possible to rotate the reservoir 10, together with that of the actuating member 130, in order to detach the flexible strip. In a variant, it is possible to provide a preloaded spring connected to said flexible strip, and which is released to pull on said flexible strip when the reservoir is moved axially towards its loaded position.

The device of the invention is simple and effective. It is made up of a small number of parts and is thus inexpensive to manufacture and to assemble, and is reliable in use. It allows optimal dispensing of the powder due to the orbital movement of the reservoir during actuation, while guaranteeing the integrity of the powder until it is used.

It should be noted that the inhaler may be refillable, by opening the body 110 to remove the empty reservoir and replace it with a full reservoir.

Various modifications can also be envisaged by a person skilled in the art, without going beyond the ambit of the present invention, as defined by the accompanying claims.

1-14. (canceled)

15. A dry-powder inhaler comprising:
a body containing a chamber and a mouthpiece provided with a dispensing orifice through which the user inhales,
a reservoir containing a dose of dry powder to be inhaled and comprising a lateral outlet opening, said reservoir being movable between an non-loaded position, in which it is at least partially disposed outside said chamber, and a loaded position, in which it is entirely disposed in said chamber, the diameter of said reservoir being less than the diameter of said chamber, so that, in the loaded position, said reservoir can move in said chamber, said body comprising tangential channels adapted to bring a flow of inhalation air tangentially into said chamber, which, during inhalation, generates an orbital movement of said reservoir in said chamber, in the loaded position,
an actuating member for moving said reservoir from its non-loaded position towards its loaded position,
characterized in that said reservoir includes a closure element which is independent from said body displaceable and/or deformable with respect to said reservoir between a closed position of said lateral outlet opening and an open position of said lateral outlet opening, said closure element being in the closed position when said reservoir is in the non-loaded position, and being in the open position when said reservoir is in the loaded position.

16. The inhaler according to claim 15, wherein said closure element comprises an asymmetrical radial projection.

17. The inhaler according to claim 16, wherein said closure element comprises at least one second radial projection which extends axially over part of the axial height of said closure element.

18. The inhaler as claimed in claim 15, wherein said reservoir is formed by an upper reservoir portion and a lower reservoir portion, which are assembled after filling with the dose of powder.

19. The inhaler according to claim 18, wherein said lower reservoir portion comprises said lateral outlet opening.

20. A dry-powder inhaler comprising:
a body containing a chamber and a mouthpiece provided with a dispensing orifice through which the user inhales,
a reservoir containing a dose of dry powder to be inhaled and comprising a lateral outlet opening, said reservoir being movable between an non-loaded position, in which it is at least partially disposed outside said chamber, and a loaded position, in which it is entirely disposed in said chamber, the diameter of said reservoir being less than the diameter of said chamber, so that, in the loaded position, said reservoir can move in said chamber, said body comprising tangential channels adapted to bring a flow of inhalation air tangentially into said chamber, which, during inhalation, generates an orbital movement of said reservoir in said chamber, in the loaded position,
an actuating member for moving said reservoir from its non-loaded position towards its loaded position,
characterized in that said reservoir comprises an upper reservoir portion provided with an upper lateral opening and a lower reservoir portion provided with a lower lateral opening, said upper reservoir portion being axially movable relative to said lower reservoir portion, said upper and lower lateral openings being axially offset when said reservoir is in the non-loaded position, and being aligned to form an open lateral opening when said reservoir is in the loaded position.

21. The inhaler as claimed in claim 15, wherein said body comprises two body portions, an upper body portion comprising said mouthpiece and a lower body portion comprising a cylindrical sleeve, said chamber being defined by these two body portions fixed to one another.

22. The inhaler as claimed in claim 15, wherein said mouthpiece comprises a grid upstream from said dispensing orifice.

23. The inhaler as claimed in claim 15, wherein said chamber is disposed between an inhalation air inlet and said mouthpiece.

24. The inhaler as claimed in claim 15, wherein said reservoir contains a dose of powder greater than 50 mg, in particular greater than 100 mg.

25. The inhaler as claimed in claim 15, wherein the volume occupied by said reservoir in said chamber in the loaded position is greater than 50% of the volume of said chamber.

26. The inhaler as claimed in claim 15, wherein the radial diameter of said reservoir is greater than its axial height.

27. The inhaler as claimed in claim 15, in which said actuating member is axially displaceable with respect to said body in order to displace said reservoir from its non-loaded position towards its loaded position.

28. The inhaler according to claim 27, wherein said actuating member comprises at least one oblique groove receiving at least one respective lug formed on said body or on an element which is secured to said body, in a manner such that during its axial displacement, said actuating member implements a rotation in said body.
